SEQUENCE LISTING (22 sequences)

SEQ ID NO: 1 - DNA, 18 bp, synthetic construct
  source 1..18: mol_type = other DNA
aacgggaagcccatcacc

SEQ ID NO: 2 - DNA, 18 bp, synthetic construct
  source 1..18: mol_type = other DNA
cagccttggcagcaccag

SEQ ID NO: 3 - DNA, 20 bp, synthetic construct
  source 1..20: mol_type = other DNA
tcctgcagtgccaccacata

SEQ ID NO: 4 - DNA, 20 bp, synthetic construct
  source 1..20: mol_type = other DNA
tggccaccatcaaaggagat

SEQ ID NO: 5 - DNA, 23 bp, synthetic construct
  source 1..23: mol_type = other DNA
tgagaagcctaagaacgcaattc

SEQ ID NO: 6 - DNA, 17 bp, synthetic construct
  source 1..17: mol_type = other DNA
cccttcgcagccatgtg

SEQ ID NO: 7 - DNA, 19 bp, synthetic construct
  source 1..19: mol_type = other DNA
ttaacgcgcagatcatgca

SEQ ID NO: 8 - DNA, 20 bp, synthetic construct
  source 1..20: mol_type = other DNA
ggtggcgttgagattgttca

SEQ ID NO: 9 - DNA, 25 bp, synthetic construct
  source 1..25: mol_type = other DNA
cgagatatacgcaggagaggtaaga

SEQ ID NO: 10 - DNA, 22 bp, synthetic construct
  source 1..22: mol_type = other DNA
gctcgacaatgttctccagctt

SEQ ID NO: 11 - DNA, 21 bp, synthetic construct
  source 1..21: mol_type = other DNA
tcacagtggttcgagcttcag

SEQ ID NO: 12 - DNA, 21 bp, synthetic construct
  source 1..21: mol_type = other DNA
gcaaacgagacatcataggca

SEQ ID NO: 13 - DNA, 23 bp, synthetic construct
  source 1..23: mol_type = other DNA
ctccgacttgtgaagtggtatag

SEQ ID NO: 14 - DNA, 19 bp, synthetic construct
  source 1..19: mol_type = other DNA
cttcctccagttgccttct

SEQ ID NO: 15 - DNA, 20 bp, synthetic construct
  source 1..20: mol_type = other DNA
cgctgagtacgtcgtggagt

SEQ ID NO: 16 - DNA, 19 bp, synthetic construct
  source 1..19: mol_type = other DNA
agagggggcagagatgatg

SEQ ID NO: 17 - DNA, 21 bp, synthetic construct
  source 1..21: mol_type = other DNA
cggcaatgaaacaaaaggcag

SEQ ID NO: 18 - DNA, 24 bp, synthetic construct
  source 1..24: mol_type = other DNA
ggctatggttatggctatggctac

SEQ ID NO: 19 - DNA, 24 bp, synthetic construct
  source 1..24: mol_type = other DNA
gtctttgactccttgctgaatctg

SEQ ID NO: 20 - DNA, 22 bp, synthetic construct
  source 1..22: mol_type = other DNA
cacctcatcgtcttccaagcac

SEQ ID NO: 21 - DNA, 20 bp, synthetic construct
  source 1..20: mol_type = other DNA
cggtcctctgagcctgtaag

SEQ ID NO: 22 - DNA, 20 bp, synthetic construct
  source 1..20: mol_type = other DNA
ggatctacatgcgacgacaa

(19) United States
(12) Patent Application Publication
(10) Pub. No.: US 2025/0002846 A1
(43) Pub. Date: Jan. 2, 2025

(54) METHOD FOR CONSTRUCTION OF INNER EAR ORGANOID

(71) Applicants: INDUSTRY FOUNDATION OF CHONNAM NATIONAL UNIVERSITY, Gwangju (KR); AJOU UNIVERSITY INDUSTRY-ACADEMIC COOPERATION FOUNDATION, Gyeonggi-do (KR)

(72) Inventors: JANG HO KIM, Gwangju (KR); YUN HOON CHOUNG, Seoul (KR); SUN HO PARK, Gwangju (KR); YEON JU KIM, Gyeonggi-do (KR)

(21) Appl. No.: 18/711,270

(22) PCT Filed: Dec. 23, 2022
(86) PCT No.: PCT/KR2022/021169

§ 371 Date: May 17, 2024

(30) Foreign Application Priority Data
Jan. 12, 2022 (KR) 10-2022-0004739

(51) Int. Cl. C12N 5/0793; C12M 1/32

(52) U.S. Cl. CPC C12N 5/062; C12M 23/12; C12N 2501/115; C12N 2501/155; C12N 2513/00; C12N 2533/54

(57) ABSTRACT

In a method for producing an inner ear organoid, stem cells and a culture medium are treated on a microwell plate whose an inner circumferential surface has a curved surface inclined downward, the stem cells are seeded in each microwell, and the stem cells seeded in each microwell are cultured. Using the method, cell aggregates for production of inner ear organoids may be easily formed, and uniform and mature inner ear organoids may be produced using the specific initial cell number control method and growth factor treatment method of the present invention, and may be used for inner ear disease modeling and drug screening.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2022/021169, filed Dec. 23, 2022, which claims priority to the benefit of Korean Patent Application No. 10-2022-0004739 filed in the Korean Intellectual Property Office on Jan. 12, 2022, the entire contents of which are incorporated herein by reference.

### REFERENCE TO AN ELECTRONIC SEQUENCE LISTING

A sequence listing electronically submitted on May 17, 2024 as a XML file named 20240517 LC0842413_TU_SEQ.XML, created on May 17, 2024 and having a size of 20,436 bytes, is incorporated herein by reference in its entirety.

### BACKGROUND

### 1. Technical Field

The present invention relates to a method for producing an inner ear organoid, which may be used in the fields of developing new drugs, treating diseases and developing artificial organs relevant to the inner ear.

### 2. Background Art

The inner ear is an epithelial sensory organ made up of interconnected hair cells, supporting cells and nerve cells, and is capable of detecting sound through the movement of sensitive stereocilia. Genetic mutations, infection, noise, aging, and external shocks cause hearing loss or balance disorders. In particular, hearing impairment that may occur with age is estimated to be about 1 billion people worldwide. However, since causes thereof are complex and research platforms are limited, there are difficulties in developing therapeutics.

Currently, several research model platforms (e.g., in vitro tissue models, cell models, and in vivo models) are being used for hearing loss research, but it is difficult to reproduce the characteristics of the inner ear structure and the function of sensory receptors, such that an efficient research model platform to replace the same is required.

Three-dimensional (3D) organoids that are mimic the multicellular composition, structure and function of organs in vivo have been introduced in developmental biology and disease mechanism studies using stem cells, which are expected to overcome the limitations of existing research model platforms.

In the case of organoids, 3D culture of stem cells with differentiation ability is required to build various cell structures like real organs, and growth factors are sequentially added in accordance with the steps in the organoid production process to control the signal transmission pathway so as to be differentiated into specific cells. Since the organoids are a platform in which cells are determined in a non-linear state like the real organs, the initial state before organoid differentiation (e.g., stem cell aggregates) is very important for the organ-specific structure and function of the final organoid.

Accordingly, in the present invention, a new method for producing an inner ear organoid using a microwell plate was developed, and the effect obtained by preparing the inner ear organoid through specific initial cell number setting and growth factor control was identified, thus the present invention has been completed on the basis of the results.

### SUMMARY

An object of the present invention is to provide a method for producing an inner ear organoid.
1. A method for producing an inner ear organoid, including: treating stem cells and a culture medium on a microwell plate whose an inner circumferential surface has a curved surface inclined downward, and seeding the stem cells in each microwell; and culturing the stem cells seeded in each microwell.
2. The method for producing an inner ear organoid according to the above 1, wherein the microwell has a width of 100 to 1000 μm.
3. The method for producing an inner ear organoid according to the above 1, wherein the inner circumferential surface of the microwell has a first curved surface inclined downward and a second curved surface inclined downward which is connected to the first curved surface.
4. The method for producing an inner ear organoid according to the above 1, wherein the microwell has a width of 450 to 550 μm, and 1.5×10³ to 4.5×10³ stem cells are seeded in each microwell.
5. The method for producing an inner ear organoid according to the above 1, further including: treating Matrigel on day 1 of culture of the stem cells; and treating bone morphogenetic protein 4 (BMP4) and SB-431542 on day 3 of culture.
6. The method for producing an inner ear organoid according to the above 1, further including: differentiating the stem cells into non-neural ectoderm; and isolating the non-neural ectoderm from the microwells.
7. The method for producing an inner ear organoid according to the above 6, further including: treating the isolated non-neural ectoderm with fibroblast growth factor 2 (FGF2) and LDN193189.

According to the method of the present invention, stem cell aggregates suitable for producing inner ear organoids may be uniformly and easily formed.

According to the method of the present invention, inner ear organoids with high maturity and improved uniformity may be prepared.

### BRIEF DESCRIPTION OF THE DRAWINGS

FIGS. 1A-1L show production of inner ear organoids, wherein FIG. 1A shows a schematic diagram of the formation of embryonic stem cell (ESC) aggregates in the prepared microholes; FIG. 1B is a representative image of retained ESCs; FIG. 1C is a representative scanning electron microscope (SEM) image of a microhole chip; FIG. 1D is an image showing that ESCs are formed over time in the microhole chip; FIG. 1E is a representative immunofluorescence image of ESC aggregates according to different initial cell numbers; FIG. 1F illustrates the quantification of an aggregate area by varying the initial cell number for 6 hours, 12 hours, 1 day and 2 days, respectively; FIG. 1G illustrates the quantification of a final aggregate area with different initial cell numbers on day 3; FIG. 1H illustrates the quantification of a time-dependent aggregate index according to the initial cell number; FIG. 1I is representative immunofluorescence images of ESC aggregates according to the initial cell number on day 3 wherein the aggregates showed different cellular structures and compaction; FIG. 1J shows the normalized fluorescence intensity measured using integrin beta 4 expression; FIG. 1K illustrates the quantification of ESCs density and area in the aggregates; and FIG. 1L is a schematic diagram illustrating effects of different aggregate sizes with different cell compaction on inner ear organoid formation.

FIGS. 2A-2H show microhole chip-based ectodermal formation of mouse ESCs with different initial cell numbers, wherein FIG. 2A illustrates a schematic diagram of ectodermal formation from ESCs; FIG. 2B is representative microscopic images of ESCs before (day 1)/after (day 3) ectodermal formation; FIG. 2C is representative scanning electron microscopy images of ESCs before and after ectodermal formation; FIG. 2D illustrates the quantification of cell density and area on the surface of mESC aggregates on day 1; FIG. 2E illustrates the quantification of surface roughness of ESC aggregates before and after ectodermal formation; FIG. 2F is representative immunofluorescence images of ESCs before and after ectoderm formation; FIG. 2G illustrates results of Western blot analysis of laminin beta 1, connexin 43, cyclin D1 and beta-actin before and after formation of ESC aggregates; and FIG. 2H summarizes initial cell number-dependent effects on ectodermal formation and stem cell pluripotency.

FIGS. 3A-3G show microhole chip-based inner ear organoid (IEO) formation of mouse ESCs performed with different initial cell numbers, wherein FIG. 3A is a schematic diagram of the micro-hole chip-based development from ESC to IEO; FIG. 3B is representative microscopic images illustrating IEOs grown in microholes at various times (days 1, 3, 5, 8, 10, 13 and 15); FIG. 3C summarizes initial cell number-dependent effects on IEO development; FIG. 3D is a representative image of IEO for different initial cell numbers on day 14; FIG. 3E is representative immunofluorescence images of ESCs before and after ectoderm formation; and FIG. 3F is merged representative high-magnification immunofluorescence images of IEOs formed in each U well plate and microhole chip with large (L) cell number, showing hair cell vesicle formation; and FIG. 3G illustrates the quantification of the number of aggregates on day 14, area of organoids, and number of buds/vesicles on day 30. “UO”=U-Org, “SO”=MS-Org, “MO”=MM-Org, “LO”=ML-Org.

FIGS. 4A-4I show effects of microhole chips on the development of inner ear organoids (IEOs) with uniformity and maturity, wherein FIG. 4A is a representative image of IEO on a U well plate and microhole chip showing the uniformity of the IEO; FIG. 4B is representative microscopic images showing IEOs grown on microhole chips and U well plates on day 1 and day 30; FIG. 4C illustrates the quantification of the perimeter and area of the IEO. “UO”=U-Org, “MO”=ML-Org; FIG. 4D is representative scanning electron microscopy images of outer and torn surfaces of IEOs grown on U well plates and microhole chips on day 30; FIG. 4E is representative immunofluorescence images of IEOS grown on each U well plate (top) and microhole chip (bottom) showing inner ear hair cells (Myo7A), supporting cells (SOX2), and DAPI; FIG. 4F is representative transmission electron microscopy (TEM) images of IEO vesicles grown on microhole chips and U well plates on day 30, and in this regard, mature kinocilia emerge from IEOs grown on the microhole chips, and the double-headed arrow indicates the length of the cilia; FIG. 4G is a representative TEM image of a cross-section of motile cilia of IEOs grown on the microhole chip; FIG. 4H illustrates the quantification of microvilli and cilia length in IEO. “UO”=U-Org, “MO”=ML-Org; and FIG. 4I summarizes the effects of microchips on the maturation of IEOS.

FIGS. 5A-5L show effects of the microhole chip on the electrophysiological function of hair cell-like cells in M-IEO, wherein FIG. 5A is a schematic diagram of membrane current analysis of hair cell-like cells in IEO; FIG. 5B is representative microscopic images of hair cells, ESCs and hair cell-like cells in IEO; FIGS. 5C-5F illustrate patch-clamp recordings of activation ranges and potassium currents of rat organ of Corti-derived hair cells, ESCs, cells in IEOs grown on U well plates and microhole chips, and show rapid internal Na+ current confirmation; FIG. 5G is a schematic diagram of cisplatin-based impaired IEO; FIGS. 5H-5J illustrate patch-clamp recordings of activation ranges and potassium currents of hair cells similar to those in IEOs grown on U well plates and microhole chips; and FIGS. 5K-5L illustrate Myo7a, oxidative stress and inflammation-related genes of IEOs grown on U well plates and microhole chips and PCR analysis results of the inflammation-related genes.

### DETAILED DESCRIPTION

Hereinafter, the present invention will be described in detail.

The present invention relates to a method for producing inner ear organoids including the steps of: treating stem cells and a culture medium on a microwell plate whose an inner circumferential surface has a curved surface inclined downward, and seeding the stem cells in each microwell; and culturing the stem cells seeded in each microwell.

The inner ear organoids (IEO) refer to an analog of the inner ear produced by culturing or recombining stem cells in three dimensions, and are used for the purpose of developing new drugs, treating diseases, and developing artificial organs.

The stem cells are stem cells that can be obtained from animals, including humans, and may be appropriately selected and used by a user without particular limitation. For example, the stem cells include embryonic stem cells (ESCs), induced pluripotent stem cells (iPSCs), adult stem cells (ASCs).

The microwell refers a groove formed on a plate, that is, grooves in a micro unit are dug on the plate so that the stem cells can be cultured.

In the present disclosure, microwells and microholes may be used interchangeably.

The microwell has a width of, for example, 100 to 1000 μm, which is remarkably small compared to the case where wells having a width of 6 to 35 mm are generally used for cell culture.

One or a plurality of microwells may be present on the plate, and for example, a plurality of wells may be spaced apart from each other and arranged at a predetermined interval.

The inner circumferential surface of the microwell may have a curved surface inclined downward, such that the inside of the well may have a hemispherical shape or a longitudinal cross-section of U-shape.

Further, the microwell may have a first curved surface inclined downward and a second curved surface inclined downward which is connected to the first curved surface, and for example, it may be a shape in which the second curved surface surrounds an inlet of the microwell having the first curved surface.

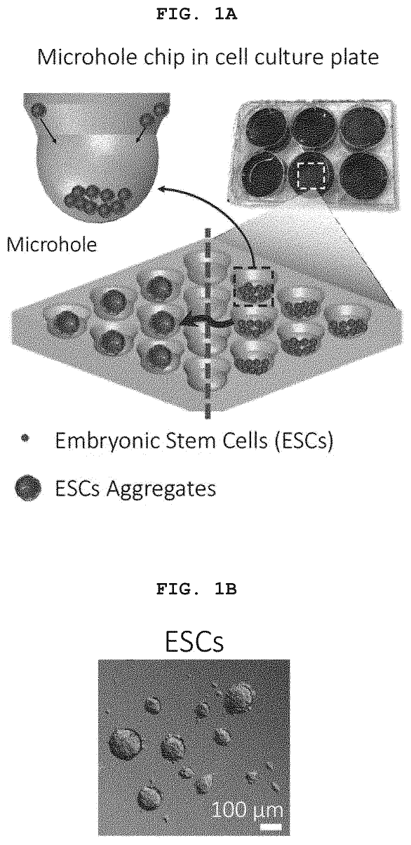

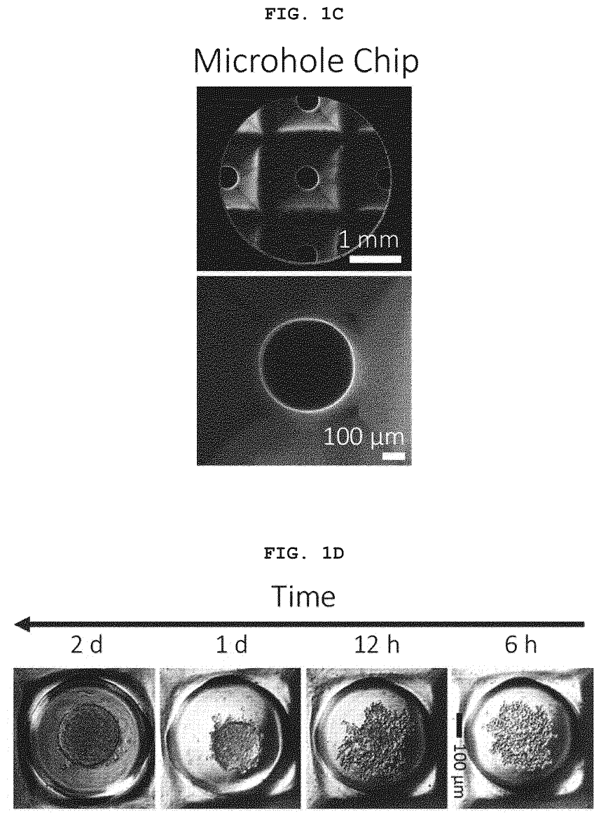

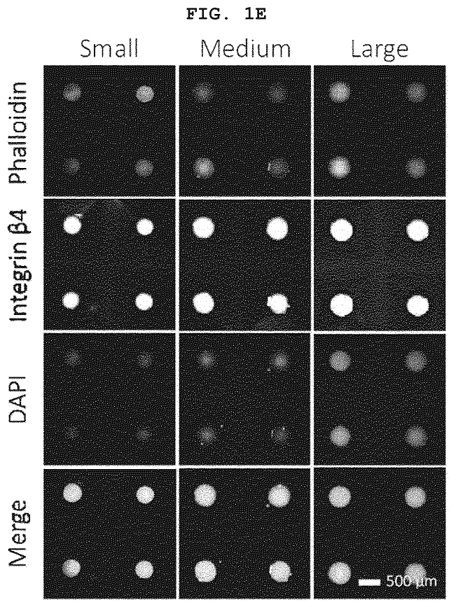

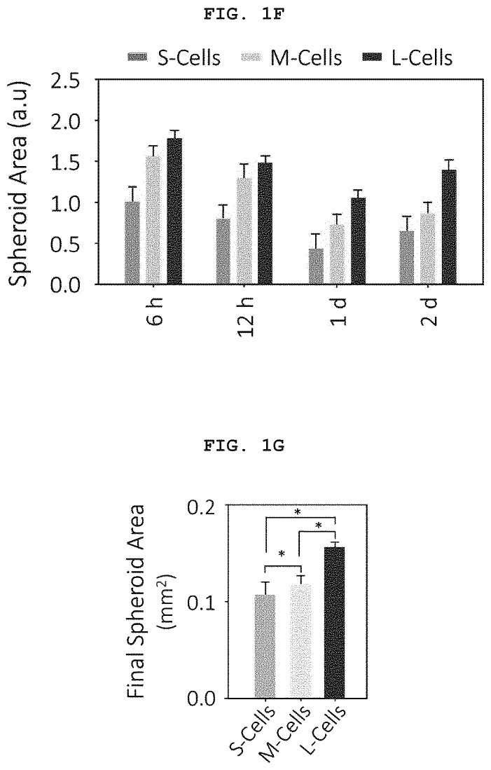

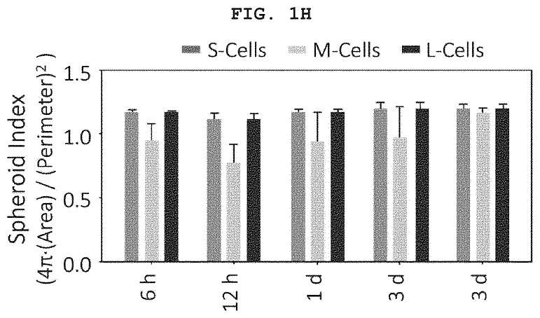

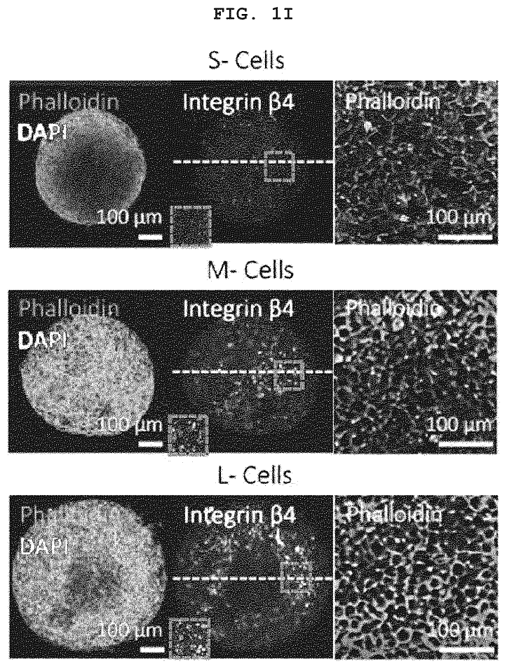

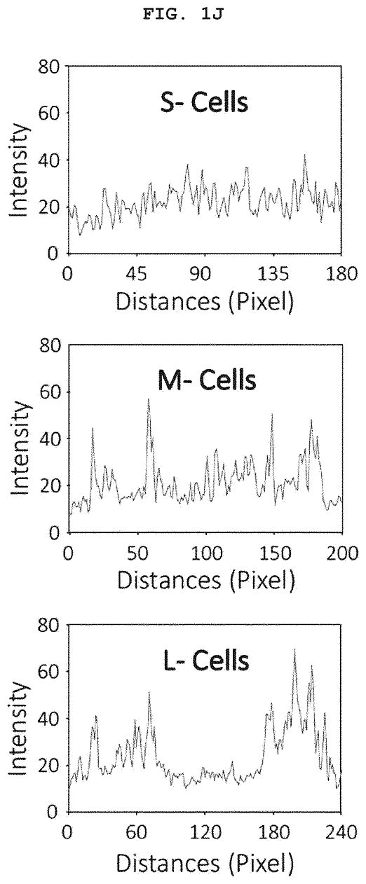

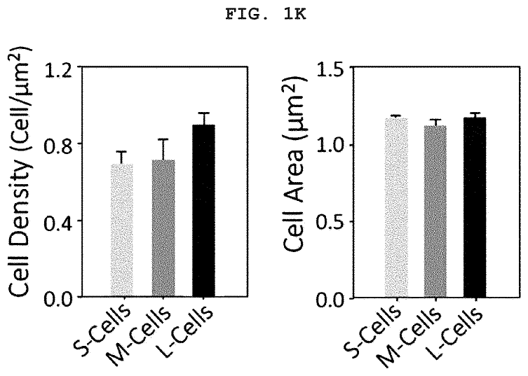

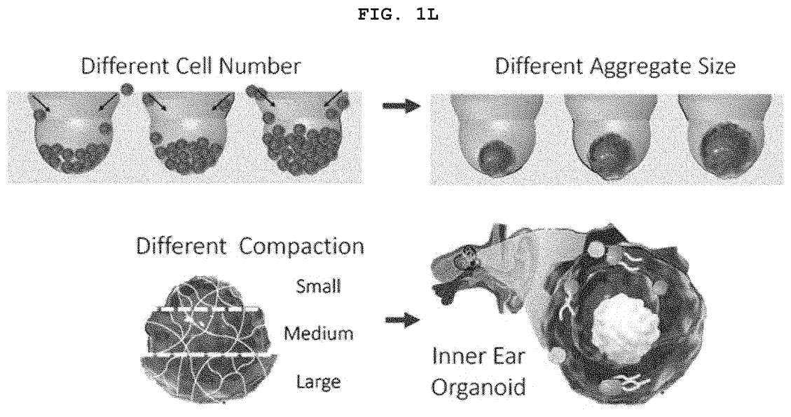

FIG. 1A shows an example of a microwell plate. As shown in FIG. 1A, the inner circumferential surface of the microwell has a first curved surface inclined downward and a second curved surface inclined downward which is connected to the first curved surface.

The seeding means dispensing stem cells into the microwells. For example, by dropping the stem cells in the form of a cell fluid onto the plate using a tool such as a pipette, the stem cells or an aggregate thereof may enter along the curved surface inclined downward of the inner circumferential surface as illustrated in FIG. 1A.

According to the method of the present invention, the seeding step is performed on the microwell plate having the curved surface inclined downward, such that stem cell aggregates suitable for production of inner ear organoids may be formed without an external force, and the inner ear organoids generated from the stem cell aggregates may be mature and produced with high uniformity.

Further, when including the second curved surface inclined downward which is connected to the first curved surface, the cells are more easily gathered to the bottom of the well including the first curved surface to form aggregates by the second curved surface in the seeding step.

In the seeding step, the number of cells seeded in each microwell may be appropriately controlled by the user according to the size of the microwell, cell type, culture conditions and the like.

For example, when the microwell has a width of 450 to 550 μm, 3.5×10³ to 4.5×10³ stem cells may be initially seeded in each microwell, specifically, when the width is 500 μm, 4×10³ stem cells may be seeded.

By controlling the size of the microwell and the number of cells, properties of the formed aggregates may be changed. For example, as the number of cells seeded in the microwells with the same size increases, the size of the formed aggregates of stem cells becomes larger, and the and integrins are better formed, a density of the aggregates may become higher.

According to an embodiment of the present invention, aggregates, which are relatively large, form more integrins and have a higher density, may increase a success rate of differentiation into inner ear organoids, and may be differentiated into mature and homogeneous inner ear organoids compared to the control which is not cultured in the microwells.

The culture means incubating the cells under artificial environmental conditions.

The culture for preparing the inner ear organoid may include seeding stem cells and then forming stem cell aggregates, which in turn are induced into definitive ectoderm (DE)-non-neural ectoderm (NNE)-pre-placodal ectoderm (PPE) in this order.

Specifically, the pre-placodal ectoderm (PPE) is a universal preplacode and includes precursors of all sensory placodes including the lens, olfactory sense, ear, and pituitary gland. The pre-placodal ectoderm may be induced into an otic placode, from which the entire inner ear will be developed.

Thereafter, the otic placode may be incorporated to form an otic pit. Then, the otic pit is separated to form an otic vesicle. The otic vesicles become mature and develop into a more complex structure, that is, the sensory epithelia.

The process of forming inner ear organoids through the culture of stem cells as described above may be performed by treatment with appropriate growth factors known to those skilled in the art to which the present invention pertains.

The growth factors for forming the inner ear organoids may be treated in a manner of adding them to a medium in the following order. For example, it may include i) treating stem cells with Matrigel in an ectodermal differentiation medium to induce definitive ectoderm, ii) treating bone morphogenetic protein (BMP) and TGF-β inhibitor to induce non-neural ectoderm, iii) treating BMP inhibitor and fibroblast growth factor (FGF) to induce non-neural ectoderm into pre-placodal ectoderm, and iv) differentiating the pre-placodal ectoderm into otic placode, followed by forming ectopic and mesenchymal epithelium in a maturation medium.

Regulation of the specific medium conditions of the above steps, the duration of each step, the concentration and amount of the growth factor to be treated, and the final step may be appropriately selected and performed by users in the field to which the present invention pertains. For example, it may be performed according to previously known protocols (see Longworth-Mills, E., Koehler, K. R. & Hashino, E. Generating Inner Ear Organoids from Mouse Embryonic Stem cells. Methods Mol Biol 1341, 391-406 (2016)).

For example, after seeding stem cells in an ectodermal differentiation medium, Matrigel is treated between 0 and 3 days of culture, which is the above step i), and BMP and TGF-β inhibitor are treated between 2 and 5 days of culture after seeding, which is the above step ii).

Specifically, the present invention may include the steps of treating Matrigel on day 1 of culture after seeding; and treating bone morphogenetic protein 4 (BMP4) and SB-431542 on day 3 of culture.

The Matrigel treatment is a factor contributing to the formation of epithelium on the surface of aggregates, and an epithelium, which is reminiscent of the final ectoderm in an embryo, may be formed immediately after Matrigel treatment. If the epithelium is not formed or is not thickened over time, Matrigel may not have been properly dissolved in the ectodermal differentiation medium. Matrigel is added to a cold medium and is necessarily admixed immediately in order to be completely mixed.

Treatment with BMP inhibits differentiation of the final ectoderm into neuroectoderm, and the BMP may be, for example, BMP-4.

SB-431542 is a type of TGF-β inhibitor, and the TGF-β inhibitor suppresses the differentiation of stem cells into mesoderm or endoderm and serves to promote ectoderm formation.

Further, the present invention may include, after step ii), separating the non-neural ectoderm from microwells and performing differentiation thereof in other wells.

That is, the present invention includes the steps of differentiating the stem cells into non-neural ectoderm, and separating the non-neural ectoderm from microwells.

As described above, after the initial culture in the microwell, a size growth of the cells is facilitated when the cells migrate to a well having a large width and then are differentiated and matured. It is known that inner ear organoids may grow up to 2 mm in size, and the size of the final aggregates may be about 10 times or more than the aggregates of the initial stem cells.

Although aggregates formed in the microwell are then separated, differentiated and matured in a general well, for example, a well with a width of 6 to 35 mm, the characteristics of the formed initial aggregates such as size, compressibility, composition, and component ratio, are the same thus to maintain the effects of the present invention.

With respect to the non-neural ectoderm of the step ii), the size of the cell aggregates is not significantly larger than that of the initial aggregates, and the surface thereof is hard enough to facilitate separation, whereby this non-neural ectoderm is suitable for separation into another well.

Although the BMP signaling mechanism is essential for the induction of the non-neural ectoderm, it should be attenuated in order to induce the pre-placodal ectoderm, which is the next stage of differentiation. Therefore, in the step iii), treating BMP inhibitor may be performed.

For example, LDN may be used as the BMP inhibitor, and specifically, LDN-193189 may be used.

The BMP inhibitor may be treated along with FGF, and the FGF may be, for example, FGF-2.

For example, the present invention include treating the separated non-neural ectoderm with fibroblast growth factor 2 (FGF2) and LDN193189.

The step iv) may include a step of treating the pre-placodal ectoderm with FGF and Wnt in order to induce the pre-placodal ectoderm into otic placode.

Co-treatment with FGF and Wnt may induce the otic placode, followed by the development of ectopic and inner ear sensory cells.

Further, after the pre-placodal ectoderm is differentiated into otic placode, it may be moved to a mature medium and cultured.

The mature medium may include, for example, Matrigel and CHIR, and specifically CHIR-99021 may be used.

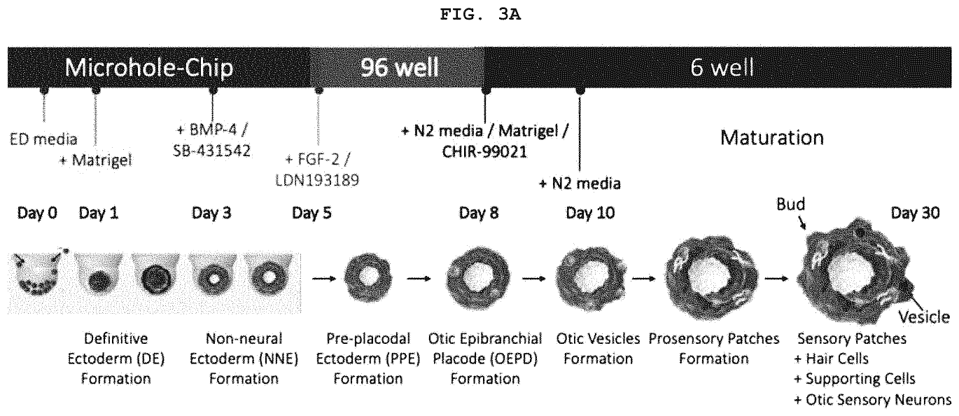

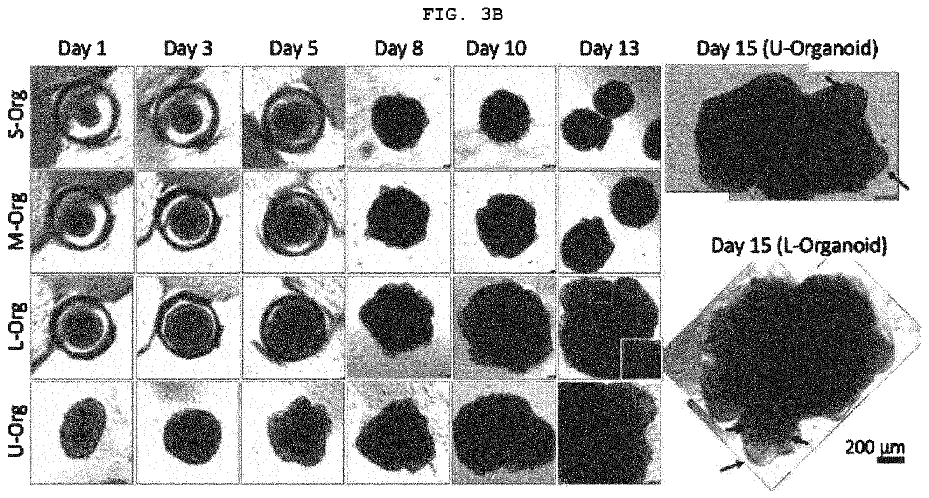

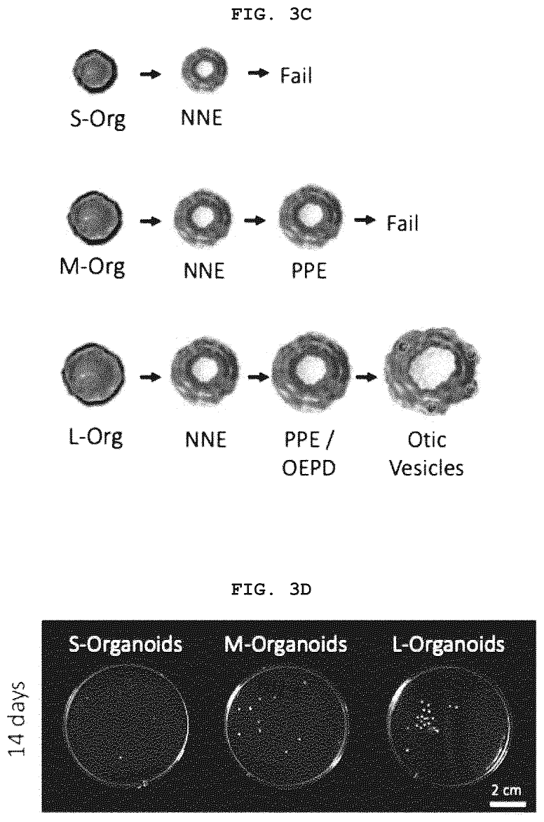

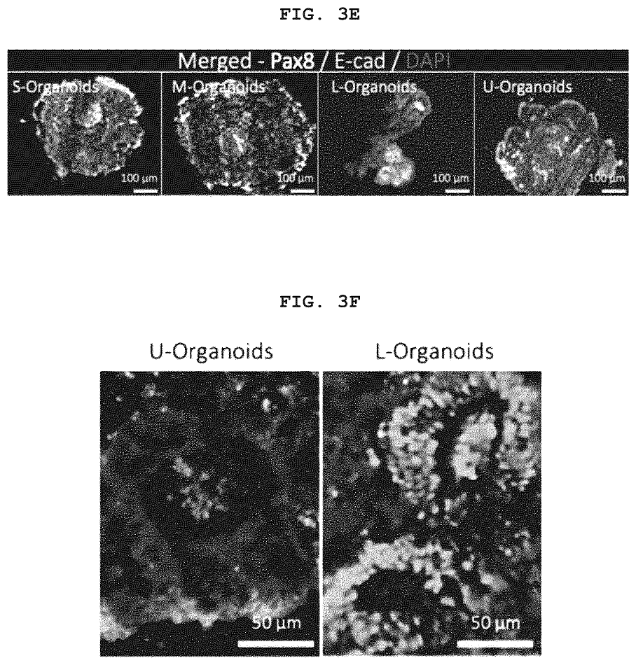

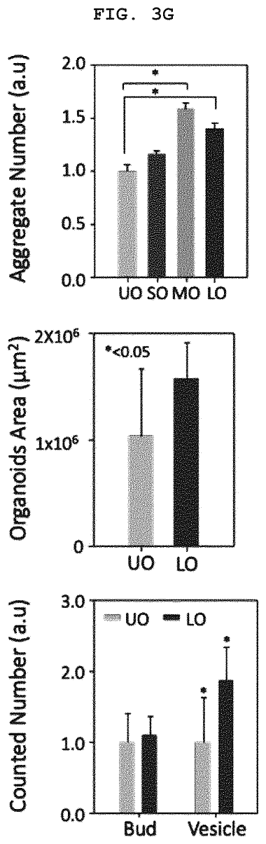

FIG. 3A of the present application schematically illustrates an example of the entire culture step, which took a total of 30 days, wherein stem cells were differentiated into non-neural ectoderm on microwells and then moved to 96 wells and 6 wells, followed by differentiation.

Hereinafter, specific examples are proposed to facilitate understanding of the present invention. However, the following examples are only given for illustrating the present invention and those skilled in the art will obviously understand that various alterations and modifications are possible within the scope and spirit of the present invention. Such alterations and modifications are duly included in the appended claims.

### EXAMPLE

### Experimental Material and Method

### 1. ESC Culture

Mouse embryonic stem cells (ESCs R1: American Type Culture Collection (ATCC), SCRC-1036) were subcultured in 0.1% gelatin-coated dishes and maintained using LIF-2i ESCs maintenance medium during expansion. All media including maintenance medium, differentiation medium and maturation medium were prepared according to previous protocols (see Longworth-Mills, E., Koehler, K. R. & Hashino, E. Generating Inner Ear Organoids from Mouse Embryonic Stem cells. Methods Mol Biol 1341, 391-406 (2016)). At 80% confluency, ESCs were subcultured again in a 1:10 split. After trypsinization, cell clumps were separated into single cells using a pipette without air bubbles in the medium.

### 2. Formation of ESC Aggregates in Microhole Chip

ESCs were dissociated into single cells using differentiation medium and washed twice to remove LiF-2i ESC maintenance medium. The ESC was counted in a microhole chip (each number of cells is 2×10³ (“Small,” “MS-Org”), 3×10³ (“Medium,” “MM-Org”), 4×10³ (“Large,” “ML-Org”)/micro-holes) with 200 μL of differentiation medium, respectively, and re-attached (50 micro-holes/12 wells). In order to remove cells remaining on the top of the microholes, the chip was carefully washed twice using a differentiation medium to remove cells remaining on the top of the microholes. 2 mL of differentiation medium was treated in each well for one day. The next day, another differentiation medium containing 2 mL of Matrigel (440 μL of Matrigel to 10.56 mL of ectodermal differentiation medium) was added to form ectoderm for 2 days. 1 mL medium in the microhole well was removed and treated with 1 mL of differentiation medium of BMP-4/SB431542 (Stemgent, Beltsville, MD, USA) for 2 days. The aggregates in microholes with 125 μL of medium were moved to a 96-well plate, and differentiation medium containing 25 μL of FGF-2 (Peprotech, Rocky Hill, NJ, USA)/LDN193189 (Stemgent, Beltsville, MD, USA) was added to each well for 3 days. Fifteen aggregates in the 96-well plate were washed with maturation medium to remove the differentiation medium, then moved to a 6-well plate without surface treatment. Then, Matrigel and 5 mL of CHIR-99021 maturation medium were added for 2 days. The maturation medium was changed every other day until day 30.

### 3. Immunofluorescence Staining

Aggregates of ESCs were immunofluorescently stained. Briefly, the aggregates were fixed with 4% para-formaldehyde (Sigma-Aldrich) for 30 minutes and washed 3 times with 1× PBS for 5 minutes. Then, samples were treated with 0.1% Triton X-100 in PBS for 30 minutes and washed three times with 1% PBS for 5 minutes each time. Thereafter, the aggregates were blocked with a 1% bovine serum albumin (BSA; GenDEPOT, Barker, TX, USA) solution for 3 hours, and then the aggregates were stained with a primary antibody diluted in BSA for 24 hours. Primary antibodies were anti-MYO7A (Proteus Biosciences Inc., Ramona; #25-6790), anti-SOX2 (Cell Signaling Technology, Danvers, MA, USA; #4900), anti-laminin β1 (Abcam, Cambridge, UK; ab44941), anti-Pax8 (Abcam, Cambridge, UK; ab53490), E-cadherin (Cell Signaling Technology, Danvers, MA, USA; #3195). After washing three times with 1×PBS, the aggregates were stained with secondary antibodies (FITC- and TRITC conjugated phalloidin, Sigma-Aldrich) diluted in PBS. After washing with 1% PBS three times for 5 minutes each, cell nuclei were stained with diluted 4,6-diamidino-2-phenylindole (DAPI; Sigma-Aldrich). Images of stained cells were obtained using a laser confocal scanning microscope system (TCS SP5/AOBS/Tandem, Wetzla, Germany). Quantification of the aggregates and cells was performed using ImageJ software (NIH, Bethesda, MD, USA).

### 4. Scanning Electron Microscopy (SEM) Imaging

Scanning electron microscopy (SEM) images of the IEO were obtained on days 1, 3 and 30 using a JSM-7500F field emission scanning electron microscope (JEOL Ltd., Tokyo, Japan). Briefly, aggregates were fixed with 2% paraformaldehyde (Sigma-Aldrich) and 2% glutaraldehyde (Sigma-Aldrich) in 0.05 M phosphate buffered saline (PBS) (Sigma-Aldrich) for 4 hours. Then, the samples were washed three times with 0.05 M PBS and finally fixed with 1% osmium tetroxide (Sigma-Aldrich) for 3 hours. Thereafter, the samples were washed three times for 10 minutes with 1× PBS and dehydrated with graded concentrations of ethanol (30%, 50%, 70%, 90% and 100%, v/v). The samples were finally coated with a Pt layer (up to 5 nm thickness) by metal sputtering and SEM images were obtained.

### 5. q-RT PCR

Total cochlear RNA was extracted using RNAiso Plus (TaKaRa, Shiga, Japan) and cDNA was synthesized using a reverse transcription kit (TaKaRa, Shiga, Japan) according to the manufacturer's instructions. qRT-PCR measurements were performed using the ABI Prism 7000 Sequence Detection System (Bio-Rad, Hercules, CA, USA) and the SYBR Green I qPCR kit (NanoHelix, Daejeon, Korea) according to the manufacturer's instructions. After normalization with GAPDH, relative gene expression was analyzed by a comparable cycle of threshold (CT) method. Expression of the gene of interest was expressed as fold change relative to the control. Both CDNA synthesis and qPCR were performed according to the manufacturer's instructions. The relative expression level of each target gene was calculated using the comparative 2-AACT method. GAPDH was used as a reference gene.

TABLE 1

| Ganas | Forward sequence | Reverse sequence |
|---|---|---|
| GADPH | 5′-AACGGGAAGCCCATCACC-3′ | 5′-CAGCCTTGGCAGCACCAG-3′ |
| | (SEQ ID NO: 1) | (SEQ ID NO: 2) |
| MYO7A | 5′-TCCTGCAGTGCCACCACATA-3′ | 5′-TGGCCACCATCAAAGGAGAT-3′ |
| | (SEQ ID NO: 3) | (SEQ ID NO: 4) |
| CAT | 5′-TGAGAAGCCTAAGAACGCAATTC-3′ | 5′-CCCTTCGCAGCCATGTG-3′ |
| | (SEQ ID NO: 5) | (SEQ ID NO: 6) |
| SOD2 | 5′-TTAACGCGCAGATCATGCA-3′ | 5′-GGTGGCGTTGAGATTGTTCA-3′ |
| | (SEQ ID NO: 7) | (SEQ ID NO: 8) |
| GPx | | |
| NRF2 | 5′-CGAGATATACGCAGGAGAGGTAAGA-3′ | 5′-GCTCGACAATGTTCTCCAGCTT-3′ |
| | (SEQ ID NO: 9) | (SEQ ID NO: 10) |
| Stat1 | 5′-TCACAGTGGTTCGAGCTTCAG-3′ | 5′-GCAAACGAGACATCATAGGCA-3′ |
| | (SEQ ID NO: 11) | (SEQ ID NO: 12) |
| IL-6 | 5′-CTCCGACTTGTGAAGTGGTATAG-3′ | 5′-CTTCCTCCAGTTGCCTTCT-3′ |
| | (SEQ ID NO: 13) | (SEQ ID NO: 14) |
| GAPDH | CGCTGAGTACGTCGTGGAGT | AGAGGGGGCAGAGATGATG |
| | (SEQ ID NO: 15) | (SEQ ID NO: 16) |
| Occludin | CGGCAATGAAACAAAAGGCAG | GCCTATCCTTATCGCTATGGCTAC |
| | (SEQ ID NO: 17) | (SEQ ID NO: 18) |
| Claudia-1 | GTCTTTGACTCCTTGCTGAATCTG | CACCTCATCGTCTTCCAAGCAC |
| | (SEQ ID NO: 19) | (SEQ ID NO: 20) |
| ZO-1 | CGGTCCTCTGAGCCTGTAAG | GGATCTACATCCGACGACAA |
| | (SEQ ID NO: 21) | (SEQ ID NO: 22) |

### 6. Patch Clamps

A 10 mV nominal increment was made at the holding potential of −84 mV covering the range of −124 to +54 mV for a duration of 170 mV, before returning to a constant potential of −44 mV (tail current, 50 ms). The bath solution was prepared using L-15 media (Gibco) and the pipette solution was prepared using 150 mM KCl, 5 mM HEPES, 0.1 mM CaCl2, 5 mM EGTA, 3.5 mM Mg ATP, pH 7.3 (KOH), and Osmolality 288 mmol/kg. To obtain damaged IEO, IEO was treated with 15 μm of cisplatin on day 30 for 48 hours (100 mg cisplatin/0.33 mL (D. W) a 3 μL cisplatin +20 mL medium). To apply the IEO for patch clamp, the IEO was treated with trypsin for 5 minutes and the detached cells were detected with a patch pipette (n=4).

### EXPERIMENT RESULT

### 1. Micro Hole Chip for Generating ESC Aggregates

For the development of microengineering-based IEO, mouse embryonic stem cells (ESCs) with different cell concentrations were cultured on a microhole chip (FIG. 1A). Suspension culture of ESCs was possible by transplanting the fabricated microhole chip to a commercial cell culture plate. The maintained ESCs were cultured in gelatin-coated well plates and had round aggregate structures (FIG. 1B). The proposed microhole chip, composed of polydimethylsiloxane (PDMS) by exquisite soft lithography, has a diameter of 500 μm for each hole under a slope that enables cell collection (FIG. 1C). Without force such as centrifugation, the cells collected in the microholes spontaneously aggregated, and FIG. 1D shows the formation of ESC aggregates over time. To determine the cell number-dependent effect of aggregates, cells were cultured at different cell concentrations (2×10³ (Small, MS-Org), 3×10³ (Medium, MM-Org), and 4×10³ (Large, ML-Org)/microholes, respectively), and maintained. After 3 days, the collected cells rapidly formed spherical structures of three-dimensional (3D) aggregates, and the size of the final aggregates varied depending on the cell concentration. In the drawings, the cell numbers “Small,” “Medium,” and “Large” are indicated by “S-,” “M-,” and “L-,” respectively.

As shown in FIG. 1E, immunofluorescence images show actin cytoskeleton and integrins of ESCs on the microhole chip. Integrin beta 4, known as a protein of the integrin extracellular matrix (ECM), and all aggregates possessed integrin ECM. The area over time and the spheroid index were quantified, and the area of the aggregates (aggregate area: 2×10³ (MS-Org) <3×10³ (MM-Org) <4×10³ (ML-Org)) (FIGS. 1F and 1G) were sensitively affected by the cell concentration. Further, the area of aggregates was decreased and then increased, demonstrating cell proliferation of ESCs after aggregation. The spheroid index indicates the spherical structure of the aggregates, and the closer to 1, the more spherical structure (FIG. 1H). The aggregates of MS-Org and ML-Org have a more rounded shape than the aggregates of MM-Org.

To confirm the effect of cell number, integrin formation and cytoskeleton of cells in the aggregates were quantified. This is because long chains of ECM fibers (e.g., integrins) are essential for rapid aggregate formation from the dispersed cells. In microholes, aggregates of different cell numbers showed different integrin formation and cell compaction (FIG. 1I). Specifically, immunofluorescence images of integrin and phalloidin expression showed increased integrin formation and cell compaction by increasing the cell number. As shown in FIG. 1J, the immunofluorescence intensities of ECM proteins of integrins are expressed higher by integral formation of ECM aggregates as the number of cells is increased. According to the quantification results (FIG. 1K), the cell density is increased with larger cell numbers (cell density: 0.009375 (MS-Org) <0.009688 (MM-Org) <0.012656 (ML-Org)/μm²—cell area (a.u.): 1 (MS-Org) >0.9724 (MM-Org) >0.95147 (ML-Org)), indicating that the larger number of cells in the microhole chip induces cell compaction.

That is, the initial cell number may influence on cell aggregates, and aggregates with larger cell numbers (i.e., 4×10³ (ML-Org)) have enhanced integrin formation and cell compaction (FIG. 1K). Therefore, it could be expected that differences in cell number regulate IEO development.

### 2. Microhole Chip to Generate ESC Ectoderm

To verify the above predictions, microengineering-based IEO ectoderm formation with different initial cell numbers was firstly investigated by Matrigel and differentiation medium. The pair of craniofacial sensory organs associated with hearing, balance, smell and vision are known to originate from ectodermal placodes near the anterior neural plate, and the ectoderm is known to be selected to form specific cranial placodes.

In general, ECM proteins (e.g., Matrigel) are used during embryonic development to promote the formation of basement membranes on the surface of aggregates that are organized into an epithelium reminiscent of the definitive ectoderm. After 1 day of seeding, Matrigel was added to the surface of each aggregate in the microholes to stimulate the organization of the definitive ectoderm (DE)

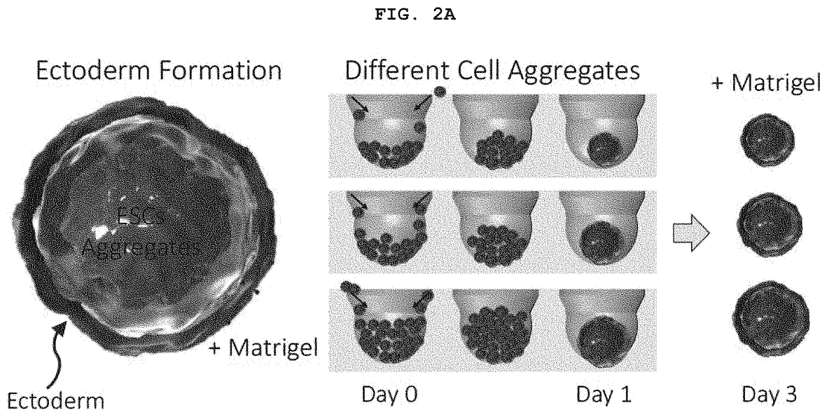

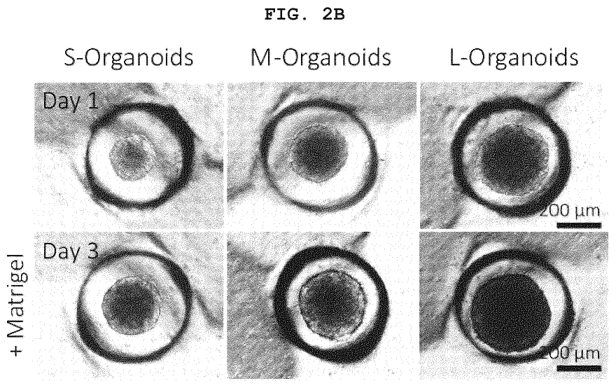

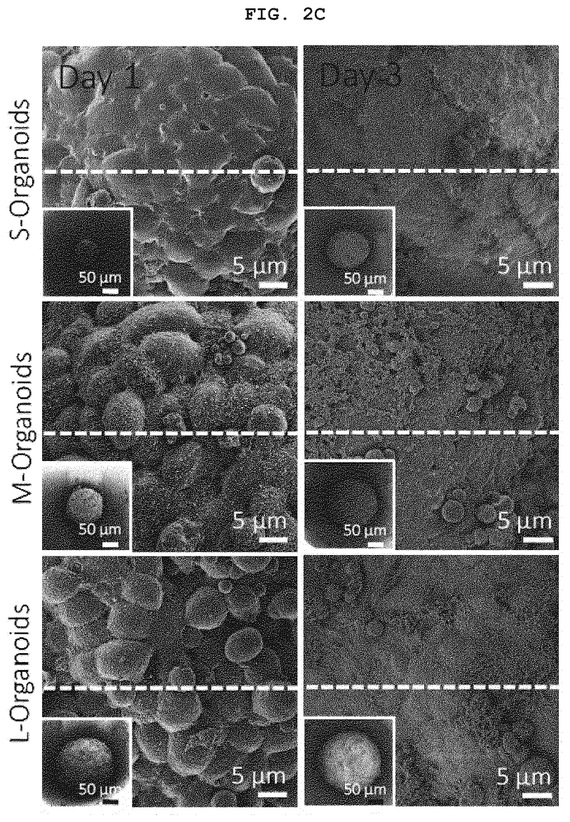

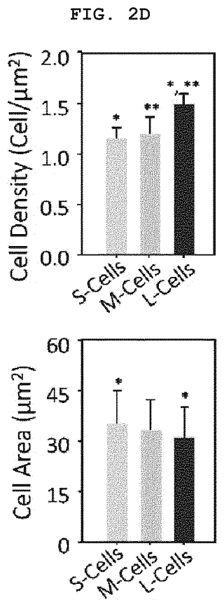

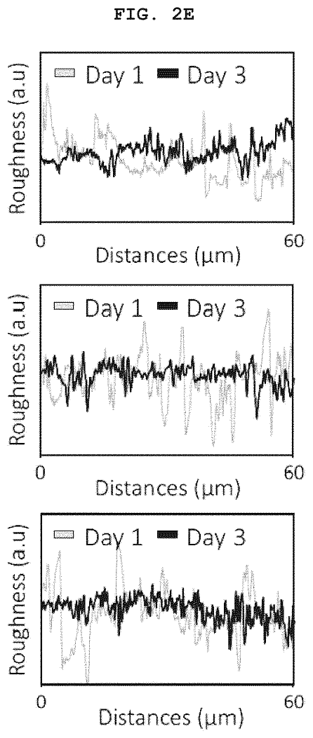

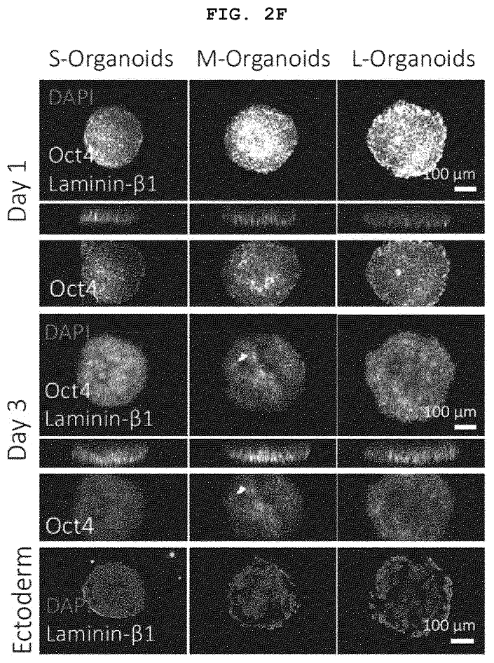

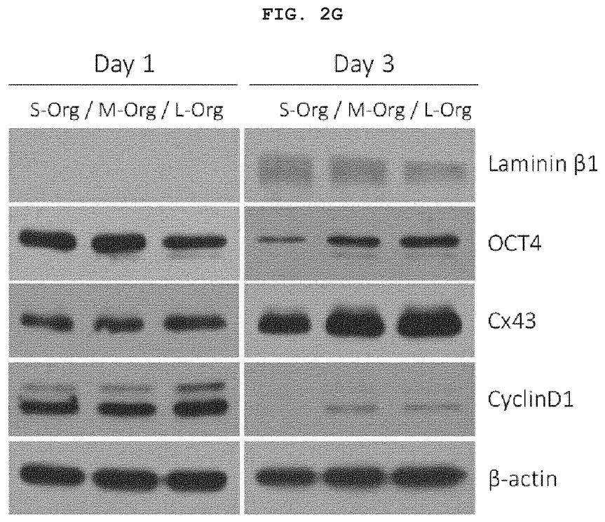

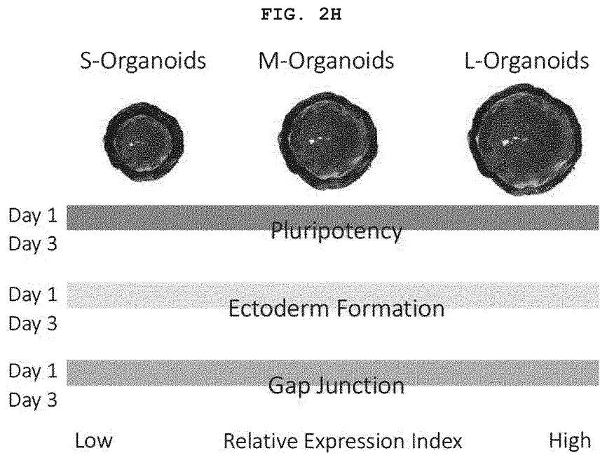

(FIG. 2A). As shown in FIG. 2B, the seeded cells with different initial cell numbers were transformed into aggregates with a thin ectodermal layer in micropores by the differentiation medium, and showed a structure of tissue with a thick layer formed on the surface after Matrigel treatment, and these different ectodermal tissues appeared while maintaining the size difference in the aggregates.

Scanning electron microscopy (SEM) images were used to compare the surfaces of the aggregates. On day 1, unlike the cell-to-cell space on the surface of MM-Org and ML-Org, the cell-to-cell space on the surface of MS-Org was almost indistinguishable (FIG. 2C), and the quantification same as area of cell density shows the trend the quantification of cell density and aggregate area in the maintenance medium (FIG. 2D). On day 3, the surface of all aggregates became smoother than that of day 1 aggregates. This is because the aggregates have definitive ectoderm including ECM components. As shown in FIG. 2E, the surface roughness was decreased on day 3 compared to day 1, and the surface roughness of MS-Org on day 1 was lower than that of other orgs (i.e., MM-Org and ML-Org). On day 1, it shows faster formation of a smooth surface at lower cell numbers.

To confirm ectoderm and pluripotency in organoids, the expression of laminin betal and Oct 4 was evaluated by immunofluorescence staining. As observed in the immunofluorescence images (FIG. 2F), organoids had ectoderm on day 3 with a slight decrease in intensity but retained pluripotency. Western blot analysis showed expression of ectoderm (laminin beta 1), pluripotency (OCT4), gap junction protein (Cx43) and cell cycle progression (cyclin D1) (FIG. 2G). From the viewpoint of determining ectoderm and pluripotency as opposite phenomena of polarity, organoids with fewer cells reduce cells with pluripotency and thus rapidly form ectoderm, whereas organoids with higher cell numbers maintain the number of cells with pluripotency and thus form ectoderm somewhat later. Further, the expression and cell cycle progression of gap junction proteins were higher as the number of cells is increased, and the expression is summarized in FIG. 2H.

### 3. Micro Hole Chip for Developing Inner Ear Organoids

During the culture of ESCs on the microhole chip, the formation of cell aggregates and ectoderm were sensitively affected by the structural stimuli of the microhole chip and cell numbers (i.e., Small, Medium, and Large). To confirm the effect of these differences on IEO development, the IEO development process on a microhole chip was optimized (FIG. 3A). Since the IEO may grow up to 2 mm, it is possible to separate the IEO from the microhole chip at the optimal point time. Various points in time were confirmed, and according to the present invention, it is appropriate to isolate the IEO on day 5 before the treatment of FGF-2 and LDN193189 after the formation of Nonneural Ectoderm (NNE), known as the surface ectoderm that produces the inner ear and epidermis.

Since the damaged IEOs may stick together, IEOs with NNEs were moved to 96 wells to increase the stability of IEOs. The following maturation process is the same as the existing technique. As shown in FIG. 3B, the aggregates were differentiated in the microhole chip for 5 days, and their morphologies were sensitively changed by treatment with growth factors. When treated with BMP-4 and SB-431542, which are NNE formation inducers and mesoderm formation inhibitors, the aggregates formed wrinkled surfaces, thereby showing the formation of thick ectoderm. These conditions were confirmed in all samples, and the size of the aggregates is increased over time. FGF-2 and LDN193189 were added to these aggregates to be used as an otic-epidermal placode inducer and BMP inhibitor, and the aggregates exhibited a thick outer skin, resulting in the formation of otic-epibranchial placode domain (OEPD). According to previous studies, aggregates have a smooth morphology with inner cells migrating to the surface surrounding the outer epithelium. However, a small number of aggregates did not form a thick outer epithelium, and on day 11 (after FGF-2 and LDN193189 treatment), differentiation did not progress gradually and cells were lost.

Cells were lost in the MM-Org aggregates, indicating weak OEPD formation, such that the next process (i.e., maturation of the IEO) could not be performed. Therefore, except for two samples of MS-Org and MM-Org, the other two samples of ML-Org and U-well organoids (hereinafter, referred to as “U-Org”) with larger cell numbers were used to analyze the microhole chip-based effect. Upon addition of the maturation medium, the aggregates showed the formation of otic vesicles with a circular ring structure along the outer margin containing sensory hair cells.

In summary, for two samples of MS-Org and MM-Org, the next step could not be performed (FIG. 3C). MS-Org could not form a smooth morphology of OEPD formation. Further, since MS-Org and MM-Org showed lower differentiation potential than ML-Org after envelop formation, it seems unlikely that the inner cells did not migrate to the outer surface or the number of cells was insufficient to perform the next step for treatment (FIG. 2G). As shown in the image of FIG. 3D, MS-Org and MM-Org did not proliferate in the aggregates and did not grow due to loss of cells. Immunofluorescence staining was performed using Pax 8 (sensory inner ear progenitors), E-cadherin (sensory outer ear epithelium), and DAPI (nucleus) to confirm specific cell expression. On day 14, the aggregates normally have otic vesicles, but the centroids retain a non-aligned structure in the inner cores of MS-Org and MM-Org, thereby indicating that mesoderm and neuronal cells have not migrated to the outer surface of the aggregates.

Unlike MS-Org and MM-Org, ML-Org and U-Org showed the formation of otic vesicles at the edges of the aggregates. In the enlarged images, it can be seen that ML-Org formed vesicles with well-aligned sensory cell structures.

### 4. Effect of Microhole Chip on Inner Ear Organoid Development

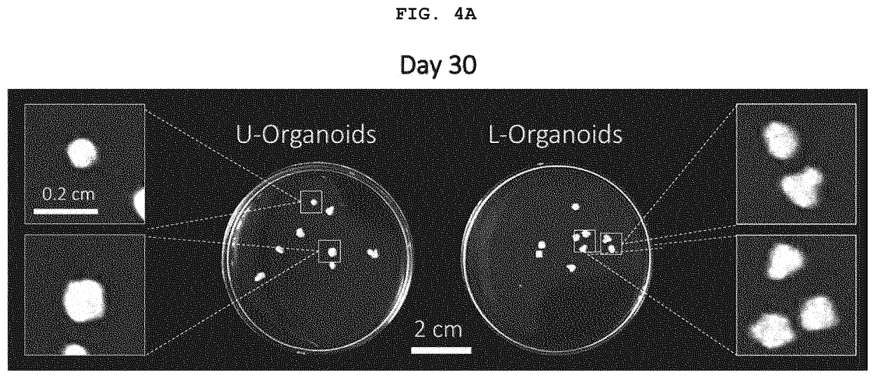

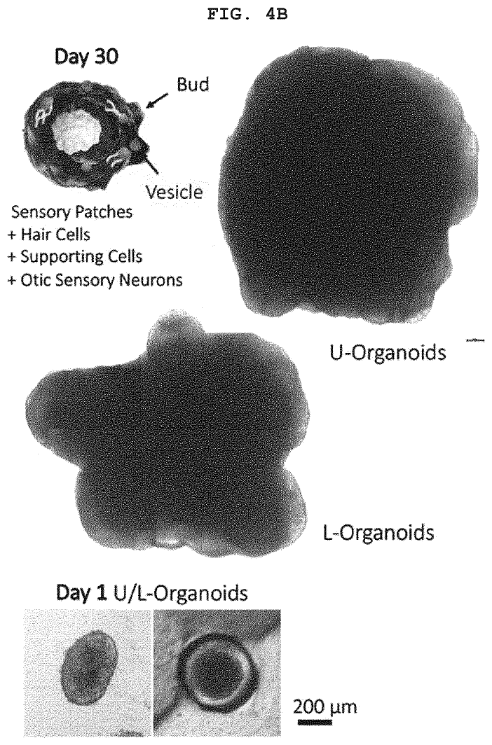

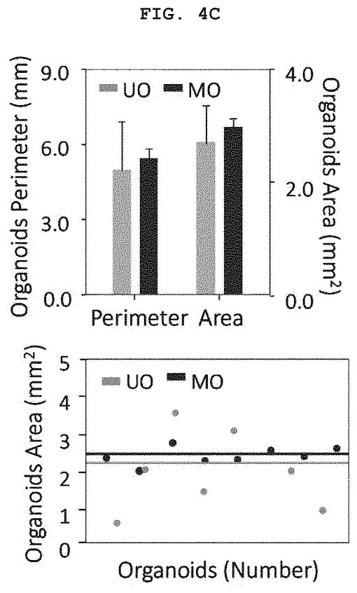

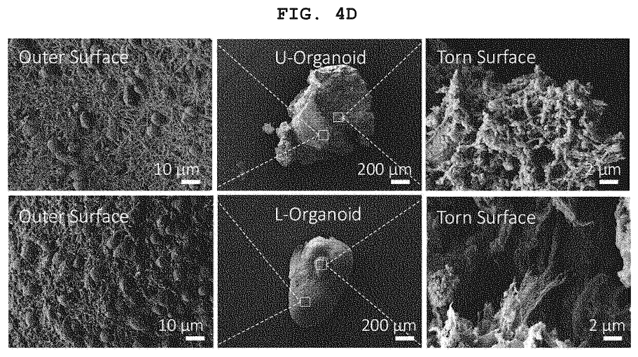

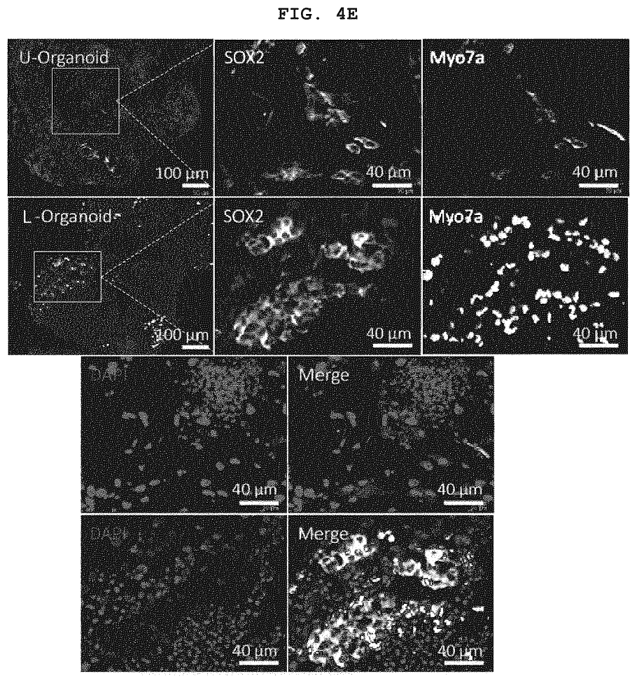

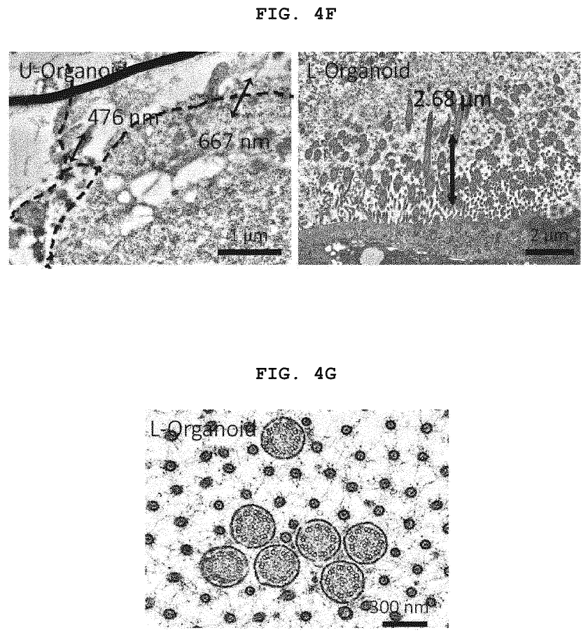

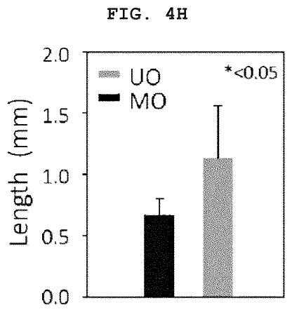

To find out whether the microhole chip has an effect on the IEO development, first, the structures of the aforementioned ML-Org (“L-Organoid” in FIG. 4) and U-Org (“U-Organoid” in FIG. 4) were compared. As shown in FIG. 4A, the initial uniformity induced the final IEO which is a clear evidence that the final IEO uniformity, uniformity was promoted by the formation of initial agglomerates in the microhole chip. The final aggregates had buds and vesicles, and the final size was more than 10 times larger than the initial size (FIG. 4B).

The final perimeter and area of ML-Org were larger than those of U-Org. In particular, ML-Org showed a similar area throughout, resulting in higher uniformity of aggregates (FIG. 4C). The final structural morphology of IEO provided additional evidence for IEO function being influenced by the initial culture conditions. Scanning electron microscopy images show the outer and torn surfaces of the IEOs (FIG. 4D). As a result of comparing high-magnification images, it could be seen that there were slight differences between U-Org and ML-Org. The outer surface of ML-Org has a dense extracellular matrix to which cells are closely connected.

Further, images of the torn surface of ML-Org show the formation of a denser extracellular matrix than that of the torn surface of U-Org.

In order to confirm the maturation of the IEO, immunofluorescence staining and transmission electron microscopy (TEM) analysis were performed to investigate the formation of sensory hair cells. Hair cells and supporting cells were visualized by immunofluorescence staining, and the expression of MyoVIIa in ML-Org was higher than that in U-Org (FIG. 4E). Surprisingly, TEM images showed the presence of hair cell motor hairs in ML-Org, unlike microvilli in U-Org that indicates immaturity of hair cells, and further showed the presence of kinocilium that has outer dynein arm, outer double lines microtubule and central single line microtubule in ML-Org (FIGS. 4F-4G). This structure shows a (9×2)+2 axoneme pattern with a unique motile ciliary structure, indicating hair cell activity. These are summarized in FIG. 4I.

### 5. Effect of Micro Hall Chip on Function

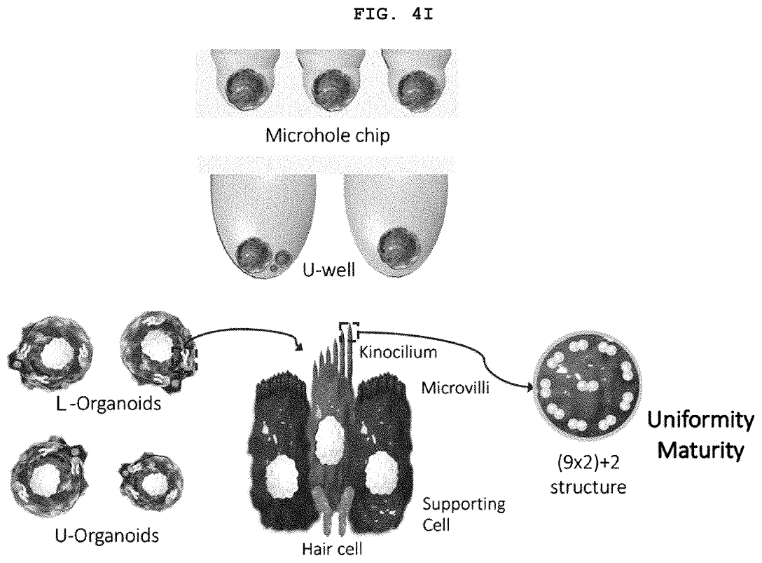

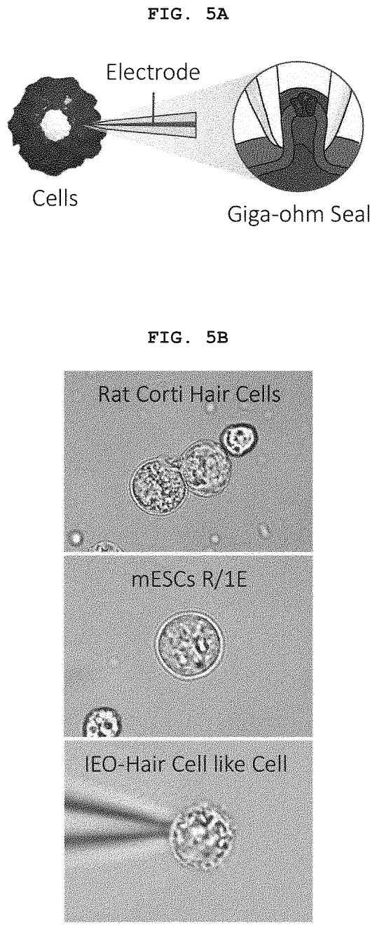

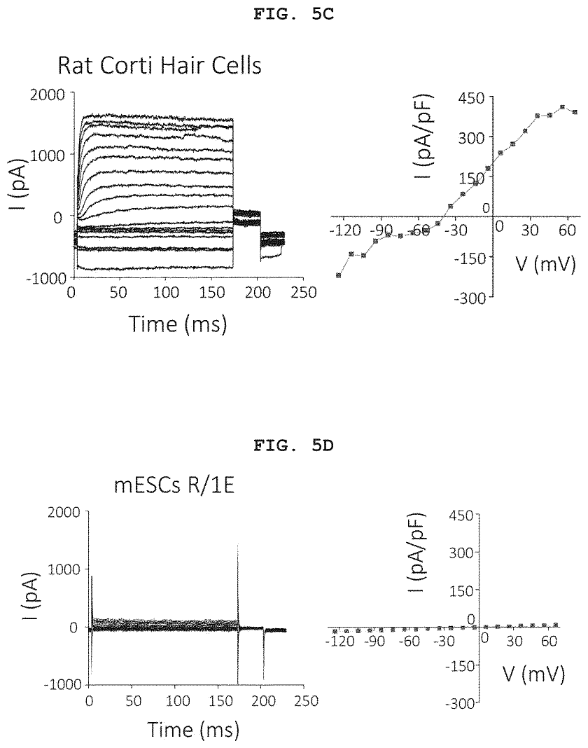

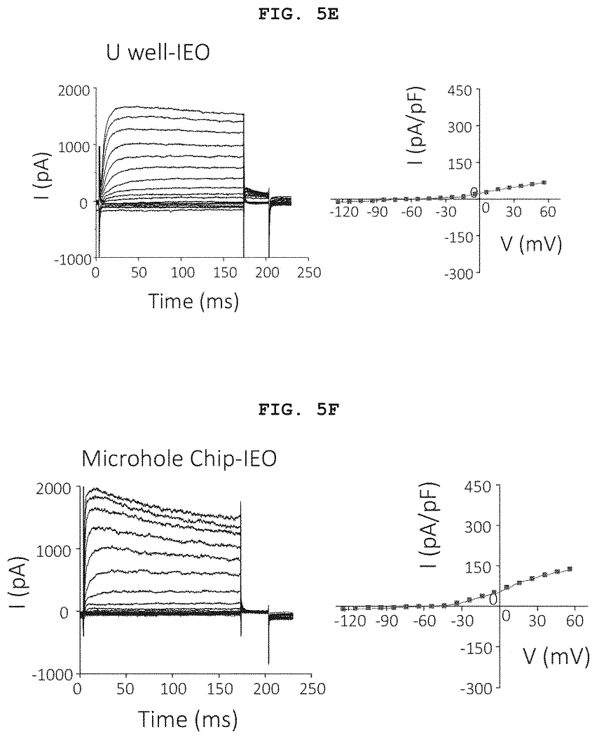

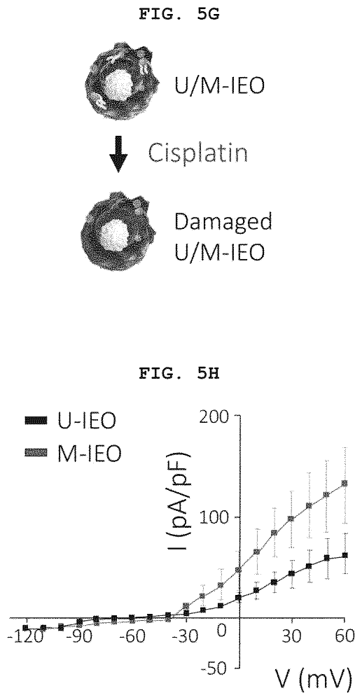

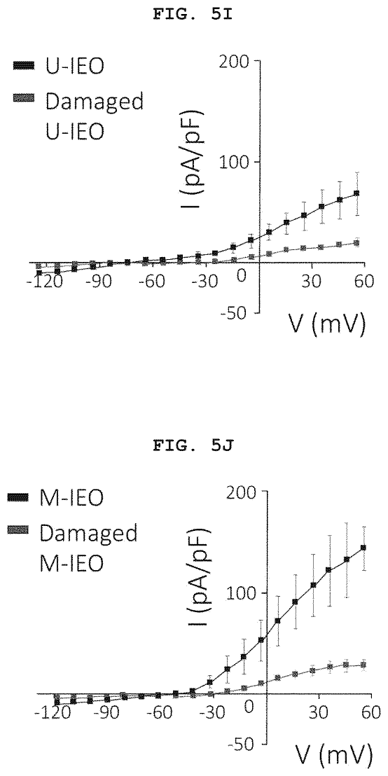

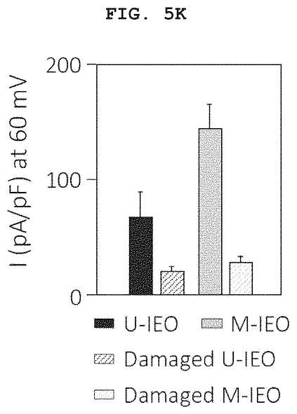

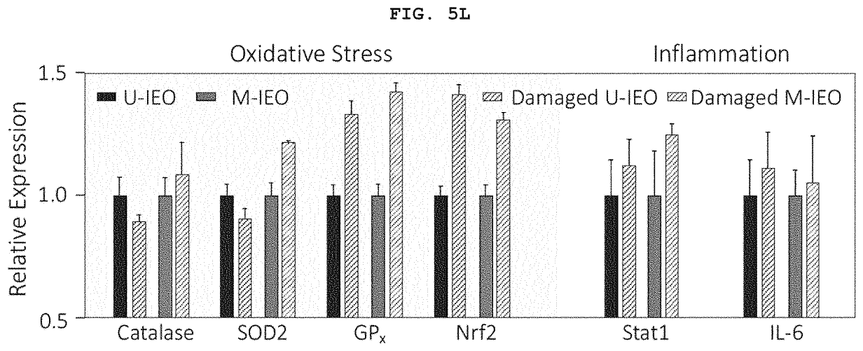

Whole cells of IEO-derived hair cell-like cells were subjected to close-seal technique, in order to compare the electrophysiology of IEOS formed in microhole chips (hereinafter “M-IEO”) and IEOs formed in U well plates (hereinafter “U-IEO”) using patch clamp assays (FIG. 5A). In the 3D structure of the IEO, cells were isolated using single cell trypsinization. First, the morphology of mouse organs of Corti hair cells, mESC (R1/E), and IEO-derived hair cell-like cells was confirmed (FIG. 5B). The isolated cells showed no significant difference according to the cell type, but the electrophysiological properties showed dramatically different results. As shown in FIG. 5C, the mouse organ of Corti-derived hair cells showed an instantaneous response by hyperpolarizing and depolarizing voltage steps, was activated up to about −45 mV, and reached a maximum size of 170 pA at −3 mV, which resulted in K+ current and external K+ current.

On the other hand, the mESCs R1/E group showed no electrophysiological function unlike the other groups, thereby indicating that undifferentiated cells could not function like hair cells (FIG. 5D). Upon increased membrane depolarization, voltage-clamp showed fast activation, rapid inactivation inward currents, possibly Na+ currents. Interestingly, hair cell-like cells derived from U-IEO and M-IEO showed an immediate response by the voltage step, and two samples were activated at around −45 mV. However, the K+ current and the external K+ current are slightly different, showing a maximum magnitude of 25 pA at −3 mV (U-Org) and 54 pA at −3 mV (M-Org) (FIGS. 5E and 5F). These results showed that M-IEO-derived hair cells were more sensitive to stimuli than U-IEO-derived hair cells. As shown in FIG. 5H, M-IEO derived hair cell-like cells have more sensitive electrophysiology than U-IEO derived hair cell-like cells with 2-fold faster activation.

As different sensitivities (i.e., function) of hair cells are expected to be affected by different drug-induced damages, and it is widely accepted that cisplatin may cause hair cell damage and hearing loss, sensitivities before/after cisplatin treatment were investigated. After 3-day culture with or without cisplatin, U-IEO-derived hair cells showed significantly reduced electrophysiology irrespective of the organoid culture technique, whereas M-IEO-derived hair cell-like cells had faster instantaneous response by voltage step than U-IEO-derived hair cell-like cells. That is, hair cell-like cells with high sensitivity have been severely damaged in a wider range (138 pA−28 pA at 60 mA=110 pA (U-IEO+Cisplatin−U-IEO)/68 pA−20 pA=48 pA at 60 mA (M-IEO+Cisplatin−M-IEO)). These results are the first study using the patch clamp system with currently prescribed drugs, and show that M-IEO may be more suitable as a research platform for complex inner ear systems for various applications such as drug screening.

1. A method for producing an inner ear organoid, comprising:
treating stem cells and a culture medium on a microwell plate whose an inner circumferential surface has a curved surface inclined downward, and seeding the stem cells in each microwell; and
culturing the stem cells seeded in each microwell.

2. The method for producing an inner ear organoid according to claim 1, wherein the microwell has a width of 100 to 1000 μm.

3. The method for producing an inner ear organoid according to claim 1, wherein the inner circumferential surface of the microwell has a first curved surface inclined downward and a second curved surface inclined downward which is connected to the first curved surface.

4. The method for producing an inner ear organoid according to claim 1, wherein the microwell has a width of 450 to 550 μm, and 1.5×10³ to 4.5×10³ stem cells are seeded in each microwell.

5. The method for producing an inner ear organoid according to claim 1, further comprising: treating Matrigel on day 1 of culture of the stem cells; and treating bone morphogenetic protein 4 (BMP4) and SB-431542 on day 3 of culture.

6. The method for producing an inner ear organoid according to claim 1, further comprising: differentiating the stem cells into non-neural ectoderm; and isolating the non-neural ectoderm from the microwells.

7. The method for producing an inner car organoid according to claim 6, further comprising: treating the isolated non-neural ectoderm with fibroblast growth factor 2 (FGF2) and LDN193189.
